(19) United States
(12) Patent Application Publication
(10) Pub. No.: US 2011/0295319 A1
(43) Pub. Date: Dec. 1, 2011

(54) DYNAMIC PEDICLE SCREW

(75) Inventors: Stephan J. Duplessis, Calgary (CA); John R. Hurlbert, Calgary (CA); Lali Sekhon, Reno, NV (US)

(73) Assignee: KINETIC SPINE TECHNOLOGIES INC., Calgary, AB (CA)

(21) Appl. No.: 13/058,623

(22) PCT Filed: Aug. 14, 2009
(86) PCT No.: PCT/CA2009/001122

§ 371 Date: May 2, 2011

Related U.S. Application Data
(60) Provisional application No. 61/189,184, filed on Aug. 15, 2008.

(51) Int. Cl. A61B 17/70; A61B 17/86

(52) U.S. Cl. 606/264; 606/305; 606/312

(57) ABSTRACT

A bone screw, such as a pedicle screw, comprises en elongate structure having a head, an anchoring portion or tip distal from the head, and an open, helical body extending there-between. In one embodiment, the invention provides a screw having an anchoring portion, which engages bone and which includes a means for engaging a driver or the like whereby the screw is driven into the bone by the anchoring portion. A method of driving a screw is also provided.

### CROSS REFERENCE TO RELATED APPLICATIONS

The present application claims priority from U.S. patent application No. 61/189,184, filed on Aug. 15, 2008, the entire contents of which are incorporated herein by reference.

### FIELD OF THE INVENTION

The present invention relates to bone anchoring devices. In particular, the invention provides an improved pedicle screw for spinal fixation.

### BACKGROUND OF THE INVENTION

Various devices and prostheses have been proposed to correct and/or stabilize spinal injuries or deformities. Such devices include artificial spinal discs, nuclei etc. Such devices serve to replace existing damaged or diseased portions of the spine. In some cases however, it is desired or necessary for fusing spinal vertebrae so as to prevent or reduce relative displacement there-between. Such fixation devices commonly utilize pedicle screws that are implanted into the pedicles of vertebrae and which serve as anchors for other prosthetic devices. FIGS. 1 and 2 illustrate a vertebral segment 1 and the pedicles 2a and 2b that extend from the vertebral body 3. FIG. 2 illustrates the placement of pedicle screws 4 as known in the art. Such screws have a threaded portion 5 that is screwed into the pedicle and a head portion 6 that connects to other fixation devices such as a rod 7.

As shown in FIG. 2, typical pedicle screw fixation systems are multi-component devices consisting of solid rods that are longitudinally interconnected and anchored to adjacent vertebrae using pedicle screws. The screws and other components are generally made of stainless steel, titanium or other acceptable implantable material. The surgeon selects from among these components to construct a system suitable for a patient's anatomical and physiological requirements. Pedicle screws are similar to the screws used in long bones.

During implantation, pedicle screws are inserted into channels that are drilled or otherwise formed through the cancellous central axis of each vertebral pedicle. The longitudinal connecting rods usually span and brace two or more vertebrae. Each vertebra typically receives a pedicle screw in both pedicles and, similarly, the connecting rods are provided in pairs each of the rods extending over one side of the spine.

Pedicle screw fixation systems have been used in providing spinal stabilization and in the promotion of spinal fusion in patients with a variety of conditions such as degenerative spondylolisthesis, isthmic spondylolisthesis, fusion after decompression, lumbar fractures, surgically repaired spinal pseudoarthroses. The advent of rigid pedicle screw/rod fixation devices has led to a dramatic increase in the rate of arthrodesis (i.e. the surgical fusion of a joint) particularly for the treatment of degenerative disc disease and spondylolisthesis. In addition to higher rates of arthrodesis, rigid instrumentation has enabled surgeons to maintain, improve, or fully reduce spondylolisthesis outright, and these devices have allowed for very aggressive strategies for decompression.

However, the use of such rigid instrumentation for the fusion of vertebrae has been associated with an increased prevalence of disc degeneration, new spondylolisthesis, disc herniation, or spinal canal stenosis at levels adjacent to the fused segments. Many surgeons suspect that the degree of stiffness of the instrumented levels relates directly to increased stress on adjacent discs and facet joints. These increased loads over time lead to segmental hypermobility, facet hypertrophy, osteophyte formation, and stenosis.

Another problem associated with current arthrodesis instrumentation is the failure of fixation of the bone screws. This problem is faced in cases of poor bone quality as in osteporotic patients. Fixation of a screw into bone is directly related to the amount of the contact area of the screw-bone interface and the quality of that contact. In other words the more direct contact there is between the bone and the surface of the screw the better the purchase or fixation. A long screw with a large diameter will provide better fixation than a short screw with a lesser diameter as a result of the larger surface contact area of the larger screw. Also the density of the bone determines the actual real contact surface between screw and bone, as bone with a high density will have more bone in direct contact with the available screw surface than bone with lower density. Thus, in patients with osteoporosis where the bone mineral density is low, there is less surface contact between the screw and bone than in patients with normal bone mineral density.

Apart from the above, other problems associated with current spine fusion instrumentation, or other orthopedic implants, relates to the loosening or breakage of the screws that are anchored into bone (Chao, C. K et al. Increasing Bending Strength and Pullout Strength in Conical Pedicle Screws: Biomechanical Tests and Finite Element Analyses. J. Spinal Disorders & Techniques. 2008. 21 (2): 130-138, 2008). Screw loosening generally occurs as a result of constant back and forth toggling forces acting on the screw such as would occur during regular flexion and extension motions of the spine. These forces result in the formation of a space between the bone and the screw and, eventually, displacement of the screw from the bone.

Shear stresses also are known to develop on pedicle screws after implantation. In these cases, once two adjacent vertebrae have been fused, they are often found to collapse or kyphose. In the result, the pedicle screws are subjected to shear stresses as the head portion of the screw is moved in a transverse direction away from the threaded portion. These stresses lead to breakage of the screws often at the connection point between the head and threaded portion.

Bone or pedicle screws currently known in the art are prone to the types of failure discussed above as they are not designed for flexibility but rather for rigidity. Examples of known pedicle screws are provided in, for example, U.S. Pat. Nos. 4,887,596 and 5,207,678. Some more recent screw and screw systems have been proposed to address some specific issues. For example, a cannulated pedicle screw is provided in US publication number 2007/0299450. In this reference, the pedicle screw is provided with a central cannula or canal having an opening at the distal tip of the screw. Once implanted, bone cement is injected into the cannula and into the joint between the screw and the bone.

U.S. Pat. No. 7,037,309 provides another cannulated pedicle screw having a self tapping distal tip. A screw of this type avoids the need for a boring hole to be provided for insertion of the screw.

US publication numbers 2005/0182409 and 2008/0015586 teach a device for dynamic stabilization of the spine and are directed to the problem of shear stresses on pedicle screws. In these references, the devices include pedicle screws that are provided with head that connects to moveable elements. In the course of regular motion, such elements are adapted to absorb compressive or expansive forces and to thereby reduce the amount of stresses translated to the screws. The moveable elements are often complicated devices as compared to the commonly known rods.

Although the above prior art examples provide improvements to specific issues, the screws taught therein all have a rigid structure. There is therefore a need for a pedicle or bone screw that would allow for the absorption and/or distribution of stresses.

### SUMMARY OF THE INVENTION

In one aspect, the present invention provides a dynamic bone screw that is sufficiently flexible for absorbing forces applied thereto while providing the necessary anchoring function.

In another aspect, the screw of the invention includes a self tapping distal tip.

Thus, in one aspect, the invention provides a bone screw having a head portion, a tip portion and a helical body extending there-between.

In another aspect, the invention provides a bone screw comprising:
an elongate body having a first end, a second end and an open helical body portion extending there-between
the first end being connected to a head and wherein the head is adapted for engaging elements of a prosthesis; and,
the second end comprising an anchoring portion for entry into a bony structure.

In a further aspect, the invention provides a bone screw comprising:
an elongate body having a first end, a second end and a body portion extending there-between;
the body portion having an open helical structure, comprising at least one open helix, forming threads on the outer surface of the body portion, wherein spaces between the threads open into an axial bore extending through the body portion;
the first end including a head; and,
the second end including an anchoring portion adapted to engage bony material.

In another aspect, the invention provides a bone screw comprising:
an elongate body having a first, proximal, end, a second, distal, end and a body portion extending there-between;
the body portion comprising an externally threaded cylindrical rod with an axial bore extending longitudinally along at least a portion thereof;
the first end including a head with an opening extending into the bore;
the second end including an anchoring portion adapted to engage bony material; and,
a first driver engaging element provided at the second end, the first driver engaging element being adapted to engage a driver for turning the bone screw.

In another aspect, the present invention provides pedicle screws.

In a further aspect, the invention provides a spinal stabilization system comprising one or more bone screws of the invention in combination with spinal stabilization prostheses, such as stabilizing rods and the like.

In a further aspect, the invention provides a method of implanting a bone screw comprising:
a) providing a bone screw having:
   an elongate body having a first, proximal, end, a second, distal, end and a body portion extending there-between;
   the body portion comprising: (i) an externally threaded cylindrical rod with an axial bore extending longitudinally along a portion of the body; or (ii) an open helix structure, wherein spaces between the threads open into an axial bore extending through the body portion;
   the first end including a head with an opening extending into the hollow cavity;
   the second end including an anchoring portion adapted to engage bony material; and,
   the second end including a first driver engaging element;
b) providing a driver having a first end adapted to engage the first driver engaging element;
c) placing the second end of the screw against a bone structure;
d) rotating the driver thereby rotating the second end of the screw; and,
e) driving the screw into the bone structure.

### BRIEF DESCRIPTION OF THE DRAWINGS

These and other features of the invention will become more apparent in the following detailed description in which reference is made to the appended drawings, which are described below. The drawings include reference numerals to identify like elements shown therein. In some cases, elements that are similar may be identified with the same reference numeral but with a letter suffix.

FIG. 1 is a schematic plan view of a vertebra illustrating the pedicles.

FIG. 2 is a cross sectional elevation of a spinal segment incorporating pedicle screws of the prior art.

FIG. 3 is a side view of a pedicle screw according to one aspect of the invention.

FIG. 4 is a side view of a pedicle screw in accordance with another aspect of the invention.

FIGS. 5 to 8 are partial side views of helical portions of the pedicle screw of the invention according to various aspects thereof.

FIG. 9 is a side view of a screw of the invention (shown in phantom) in combination with a driver.

FIG. 10a is an end perspective view taken from the distal end of the screw of FIG. 3.

FIG. 10b is a distal end view of the screw of FIG. 3.

FIG. 11a is a side view of screw of the invention according to another aspect comprised of multiple components in the assembled state.

FIG. 11b is the screw of FIG. 11a in the unassembled state.

FIG. 12 is a side view of the body portion of the screw of FIG. 11a.

FIG. 13 is a side perspective view of bone engaging element of the screw of FIG. 11a.

FIGS. 14a to 14c are side perspective views of the head of the screw of FIG. 11a.

FIG. 15 is a top view illustrating the pedicle screw of FIG. 3 implanted in a vertebra.

FIGS. 16a and 16c are side views of a pedicle screw and driver combination according to one aspect of the invention, shown in the assembled and unassembled states, respectively.

FIG. 17 is a side view of a screw of the invention showing a helix with a variable pitch.

FIGS. 18 and 19 are side views of a screw of the invention showing a helix with a variable pitch and taper.

FIG. 20 is a side perspective view of a bone engaging element according to one aspect.

FIG. 21 is a proximal end perspective view of the bone engaging element of FIG. 20.

FIG. 22 is a distal end view of the bone engaging element of FIG. 20.

FIG. 23 is a side cross sectional view along the length of a bone screw according to another embodiment of the invention.

FIG. 24 is a side view of the bone screw of FIG. 23.

FIG. 25 is a side cross sectional view of another embodiment of the head for use with the bone screw of FIG. 23.

FIG. 26 is a side view of the head of FIG. 25.

FIG. 27 is a side view of a combination of the bone screw of FIG. 23 and the head of FIG. 25.

FIG. 28 is a side view of a combination of a bone screw according to another embodiment and the head of FIG. 25.

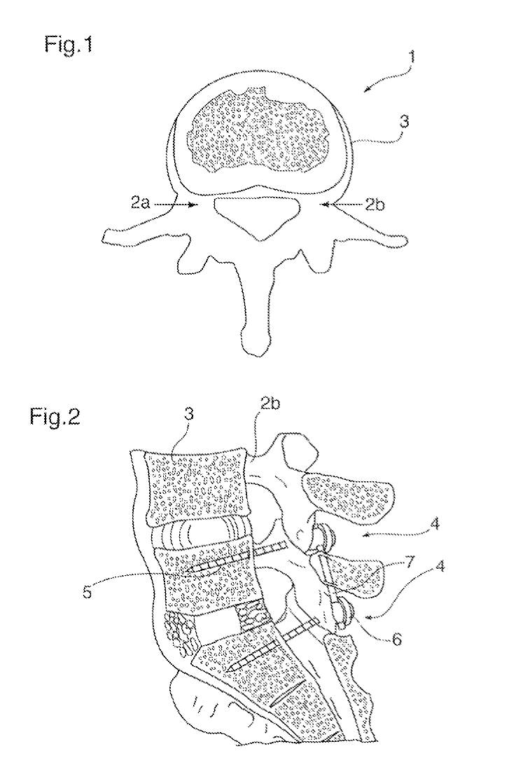

### DETAILED DESCRIPTION OF THE INVENTION

The invention will now be described with reference to various embodiments thereof. The following description will refer primarily to pedicle screws and to spinal stabilization. However, it will be understood by persons skilled in the art that the invention can be equally applied to any bone screw used in anchoring or fixation applications. Thus, the references herein to pedicle screws and/or to spinal fixation or fusion will be understood as being illustrative of a particular embodiment of aspect of the invention and are not intended to limit in any way the application of the invention in other areas of orthopedic surgery.

The invention can, for example, be used in applications involving various large bones such as the femur, tibia, fibula, ulna, etc. All references to “pedicle screws” as used herein will be understood as meaning bone screws of any type as known in the art, but adapted in the manner contemplated by the invention.

Further, unless otherwise indicated, the term “screw” will be understood to mean a unitary structure or a combination of structural units, such as a head, body and distal end, as described below.

It will be understood that the following description of the invention will be made with reference to the figures and elements shown therein and that such elements will be identified with one or more reference numerals. Unless indicated otherwise, the characteristics or features of any of the elements depicted in the figures will be understood to apply to all equivalent elements, indicted as being such, regardless of any difference in the reference numerals used to identify same. In the present disclosure, the terms “distal” and “proximal” are used to describe the screws of the invention. These terms are used for convenience only and are not intended to limit the invention in any way. As used herein, the term “distal” will be used in relation to that end of the screw of the invention that is inserted into bone. The term “proximal” will be used to refer to the opposite end of the screw that extends outside of the bone into which the screw is implanted. Thus, although these descriptive terms are used to describe the screws of the invention in reference to their placement in bone, it will be understood that the invention is not limited to screws solely when in use or solely when implanted or otherwise combined with bone.

In the present description, the terms “open helix” or “open helical structure” are used. These terms will be understood to refer to a hollow structure comprising one or more helically wound elements, resembling a “corkscrew”. The helical structure forms a continuous thread to provide the screw functionality. The outer surface of such structure may include a cutting edge for assisting in the screw function. The spaces between the threads are open to a central bore.

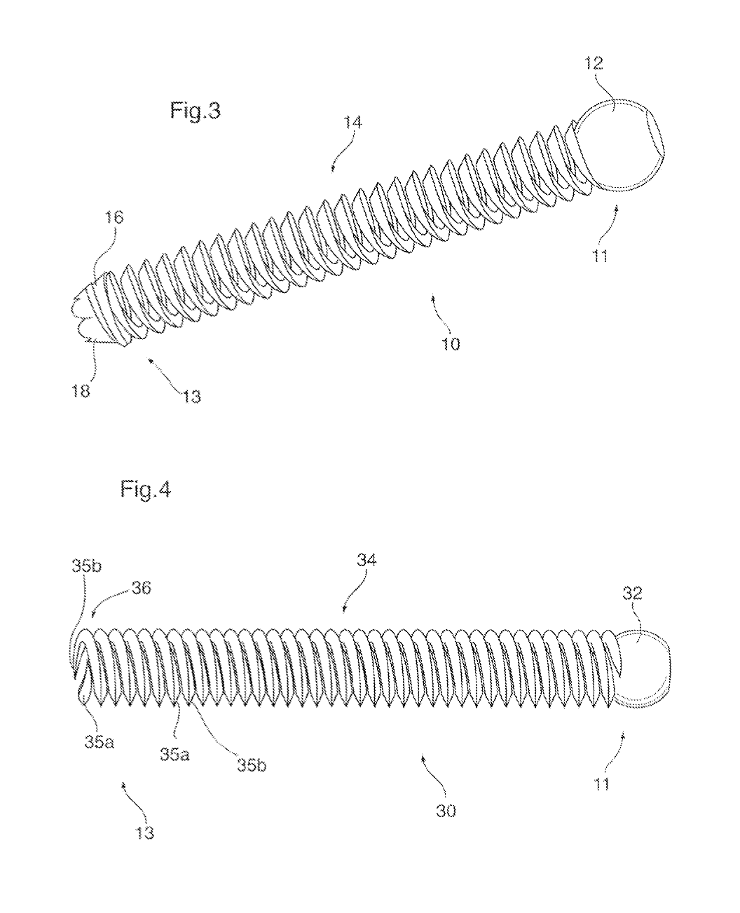

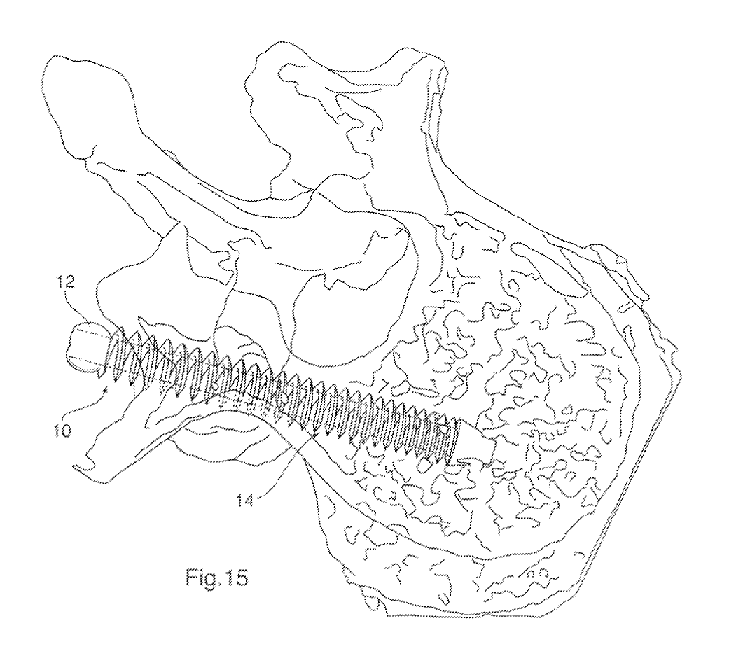

FIG. 3 illustrates a pedicle screw (or bone screw) of the invention in accordance with one aspect. As shown, the screw 10 generally comprises an elongate structure having a proximal end 11, an opposed distal end 13 and a body portion 14 extending there-between. FIG. 15 illustrates the screw 10 when implanted through a pedicle in a vertebra. The proximal end 11 includes a head 12 of the screw, which extends outside of the bone once the screw is implanted. The head 12 may be provided with any one of a variety of configurations for use in connecting the screw to other elements of a spinal stabilization system. For example, the head 12 may be provided with a yoke for receiving a rod for spinal stabilization and a locking block for locking the rod within the yoke. Such a combination is shown, for example, in U.S. Pat. No. 4,887,596. Alternatively, the head 12 may be provided with any other known or desired configuration such as, for example, taught in the references mentioned above. It will also be understood that the head 12 may also be provided with a receiving means for engaging a driver or the like (i.e. a “driver engaging element”) for rotating the screw during implantation as discussed further below. It will be understood that the invention is not limited to any specific design or configuration of the head 12.

The distal end 13 comprises the portion of the screw 10 that is inserted into the bone during implantation. The distal end is generally provided with an anchoring portion or tip 16 for engaging the bone into which the screw is to be implanted. It will be understood that although element 16 (and others as discussed below) is referred to as an “anchoring portion”, this term is used simply for convenience. Persons skilled in the art would understand that, during implantation of the screw 10, the anchoring portion 16 is the simply the first portion of the screw to be inserted into the bone in question. Upon further implantation of the screw, it will be understood that other portions along the length thereof will engage bone and will, therefore, be “anchored” therein.

The body 14 of the screw 10 comprises, in a preferred embodiment, an open helical coil shape or a helical spring shape, thereby assuming a generally “corkscrew” structure. As can be seen in the figures, the body 14, comprises a single element or thread arranged in a helical manner. Outer surface of the body thereby forms the threads of the screw. In a preferred aspect, the outer edge of the helix includes a blade or sharpened portion for engaging the bony structure into which the screw is to be implanted. The “open” nature of the body results in a hollow core as well as openings between the threading extending into the core. The term “open helix” will be used herein to refer to the structure mentioned above.

Another embodiment of the screw of the invention is shown in FIG. 4 wherein the screw 30 includes a head 32 at the proximal end 11, a body 34 and an anchoring portion 36, at the distal end 13, similar to those elements described above. However, unlike the embodiment shown in FIG. 3 wherein the screw of the invention comprises a single helix, the embodiment shown in FIG. 4 comprises a body 34 having two helical elements 35a and 35b, both coaxial with each other and both connected to a common head 32. By using a “double helix” structure for the body 34, the screw allows an even greater amount of surface area contact between the screw and the bone into which it is implanted. It will also be understood that the double helix structure also provides a screw that has greater stiffness than a single helix structure. It will be understood that, in other embodiments, a screw of the invention may comprise more than two helical elements.

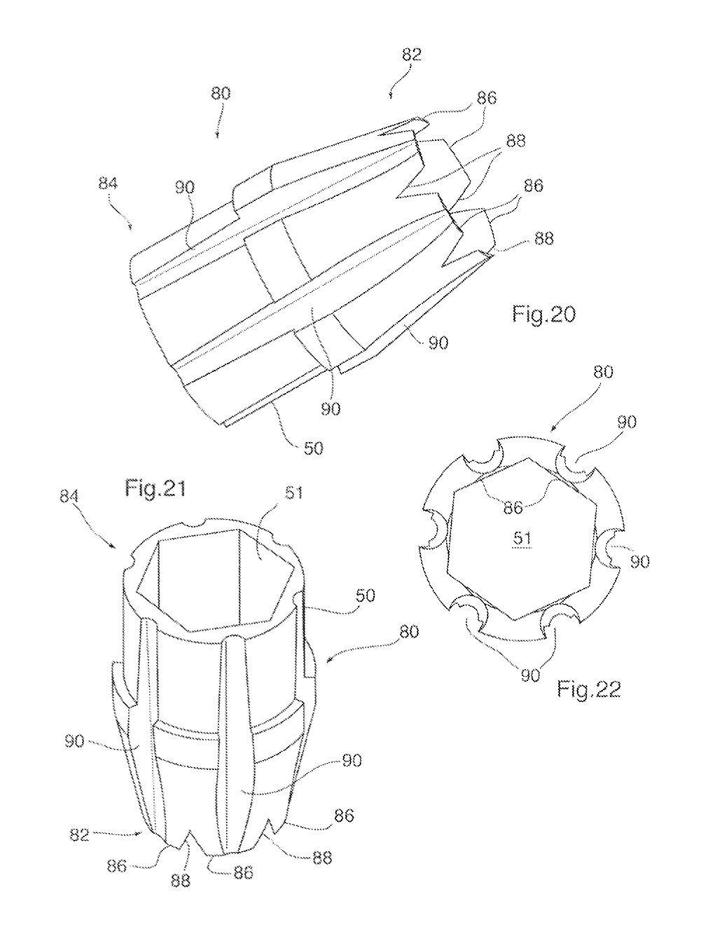

The anchoring portions 16 or 36 of the screw serve to engage the bone at the site of implantation. For assisting this function, the anchoring portions may be provided with or may comprise a point for piercing and entering the bone. In another aspect of the invention the anchoring portion 16 may be provided with a bone engaging element 18 or other similar structure to assist in the implantation of the screw. In one aspect, the bone engaging element 18 may comprise a self-tapping device, such as that taught in U.S. Pat. No. 7,037,309 or other similar structure that allows the screw to be self-boring into the bone upon being rotated. As will be understood, such a self-tapping or self-boring mechanism may obviate the need for separately boring a hole in the bone prior to implanting the screw. This aspect of the invention is discussed further below in relation to FIGS. 20 to 22.

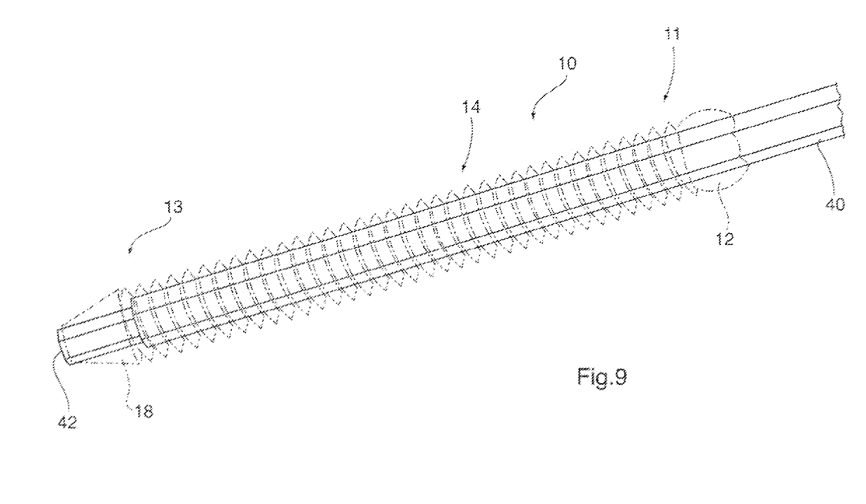

In another aspect, as discussed further below with reference to FIG. 9, the bone engaging element 18 may include a rotating means for engaging an end of a driver or the like (i.e. a “driver engaging element”). The driver may comprise any known mechanism used for implanting bone screws. In this configuration, and when the screw 10 is being implanted, the actuating end of a driver would be extended longitudinally through the center of the screw 10 and engage a cooperating structure provided by or in the rotating means of the bone engaging element 18. For example, such rotating means may comprise a hexagonal ring within the lumen of the bone engaging element 18 that is adapted to receive a cooperating hexagonal end of a driver. A driver having an actuating hexagonal head can then be inserted through the head 12 of the screw and longitudinally through the open helix of the screw. The head would then extend through and engage the hexagonal ring of the bone engaging element 18. Once engaged, rotation of the driver would serve to rotate the bone engaging element 18. Since the latter is fixedly connected to the body 14, the entire screw would thereby be rotated. It will be understood that with this arrangement, turning of the driver (not shown) will result in the screw 10 being “pulled” into the bone as opposed to being “pushed”, as would be the case if the head 12 were engaged by the driver. It will be appreciated that in this version of the invention, the head 12 would preferably include a passage through which the driver would extend. Further, although the above description is provided with reference to a hexagonal nut/driver structure, any similarly functioning structure would also be usable in the invention.

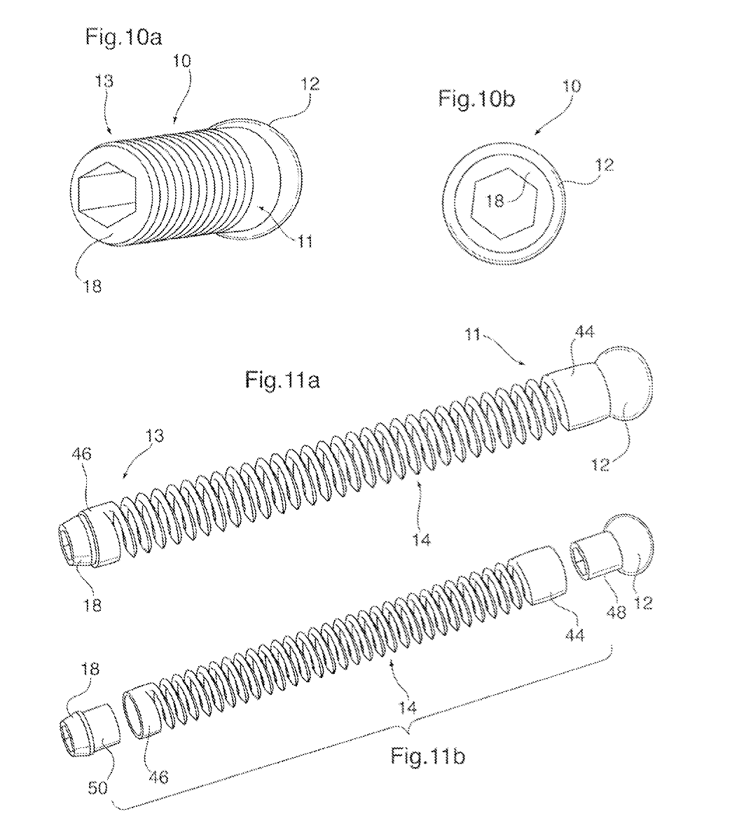

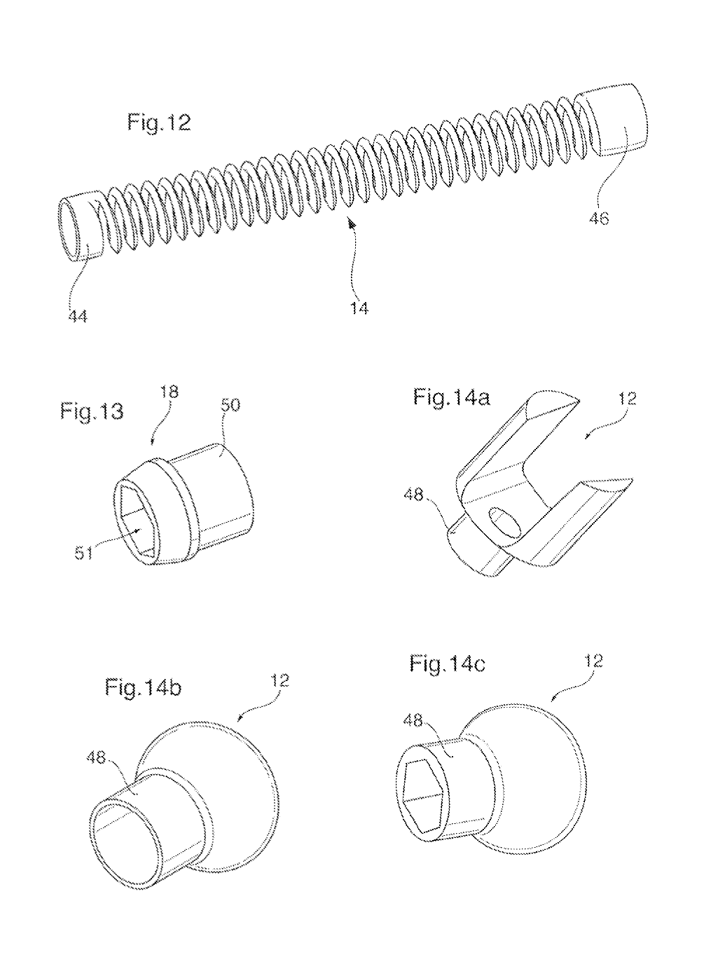

As shown in FIG. 9, a driver 40 is provided having a size capable of extending through an opening in the head 12. The distal end 42 of the driver 40 is extendable through substantially the entire length of the screw 10 and is adapted to engage, in one embodiment, the bone engaging element 18. In some cases, the distal end 42 of the driver may also extend through the bone engaging element 18. At least the distal end 42 of the driver 40 is provided with an outer surface having a geometry that functions as a drive shaft. As known in the art, the end of the driver 40 opposite to the distal end 42 may be provided with a handle or other similar structure (not shown) that facilitates rotation of the driver 40. As shown in FIGS. 10a and 10b, the bone engaging element 18 of the screw 10 includes an inner surface 44 having a geometry that is complementary to that of the distal end 42 of the driver. In the embodiment illustrated in FIGS. 9 and 10, the distal end 42 of the driver 40 and the inner surface of the bone engaging element 18 are provided with hexagonal cross section. Although such an arrangement provides an efficient means of imparting rotation force from the driver 40 to the screw 10, it will be understood that such geometry is not the sole means possible. Various other geometries will of course be known to persons skilled in the art for achieving the purpose of rotating and, thereby, driving the screw into the bone.

Although the above discussion has focused on the bone engaging element 18 being capable of engaging the driver 40, it will be understood that any similar driver engaging means or device may be provided within the body 14 of the screw 10 at either the distal end 11, the proximal end 13 or at any position there-between. Such driver engaging means may comprise an annular ring disposed co-axially within the lumen of the body 14. The outer surface of the annular ring would be secured to the inner surface of the body 14 (such as the helix portion). The inner surface of the annular ring would be provided with a geometry that is complementary to the outer surface of the driver. It will also be understood that one or more of such annular rings may be provided at various positions along the length of the body 14 or the screw 10 itself. Similarly, although reference in made to “annular rings” persons skilled in the art will understand this term to mean any type of driver engaging device. That is, a device that is capable of receiving and engaging a driver and imparting a rotational motion to the entire screw.

In a further aspect, the above described means of implanting a screw by rotation of the distal end may equally be applied to screws not having the aforementioned open helical structure. That is, the invention provides a pedicle or bone screw that comprises a solid screw similar to those known in the prior art. In this aspect, the invention provides a screw that is similar in structure to the screw 10 described above. That is, the screw would include a proximal end, with a head, an elongate body, and a distal end, preferably with an anchoring portion and/or a bone engaging element. Such screw comprises an elongate hollow or cannulated structure, wherein a central bore is provided extending through the substantial portion of the screw. The term “substantial” as used in this context refers to a bore that extends from the proximal end to at least distal end. In one case, the bore may extend through the distal end as well. The cannula of such screw is provided with a diameter that is sufficient to accommodate a driver such as that described above. The outer surface of the screw includes a thread for engaging bone upon being screwed into same. The distal end of the screw is provided with a driver engaging means as described above. In this manner, the screw can be implanted into a pedicle (or other bone structure) by rotating the driver and, thereby, “pulling” the screw into the bone. That is, the screw will be driven into the bone by rotation of the distal end as opposed to being “pushed” by rotating the proximal end.

In another embodiment, the screw may be rotated by applying the rotational force at the proximal end of the screw. For example, the head 12 of the screw may be adapted to be rotated as is commonly known in the art. In such an embodiment, any known means for rotating the head of known pedicle screws may be utilized in the invention. For example, the head 12 may be provided with any opening or structure to receive a cooperating driver. In one example, the head 12 may be provided with a female hexagonal opening, similar to that described above, into which a hexagonally shaped driver can be inserted or through which such driver can be extended. Rotation of the driver would then impart a rotational force to the head 12 and, thereby, to the screw 10. As indicated above, pedicle and other bone screws are commonly implanted using this approach of driving the screw via the head portion.

In yet a further embodiment, the screw of the invention may be driven by a single driver acting upon both the distal and proximal ends simultaneously. In this embodiment, the bone engaging element 18 and the head 12 may be provided with a rotating means to engage the same driver. For example, referring again to FIG. 9, it can be seen that the driver 40 may be provided with a smaller outer dimension at the distal end thereof as compared to the proximal end. Thus, it is possible for both the distal 13 and proximal 11 ends of the screw 10 to be driven simultaneously by the same driver 40 if both the bone engaging element 18 and the head 12 are provided with an inner engagement means for cooperating with the outer surface of the driver. A bone engaging element 18 and head 12 that are adapted for this arrangement are illustrated in FIGS. 13 and 14c, respectively. In this aspect, the insertion of the driver 40 into the head 12 of the screw 10 would not impede the travel of the driver towards the distal end of the screw.

In another aspect of the above embodiment, the driver may be of a single size, adapted to engage the bone engaging element 18. The head 12 may also be provided with an engaging surface to be acted upon by the driver. However, the opening at the head 12 may be sized larger that the exterior surface of the driver. In order for the driver to actuate the head, a sizing collar having, for example, inner and outer hexagonal surfaces adapted to fit over the driver and within the opening of the head 12, may be slid over the driver and be trapped within the opening in the head. In this way, the driver may be used to initially rotate only the distal end of the screw and, later and/or when necessary, rotate both the distal and proximal ends. As will be understood, various other combinations of this feature may be used so as to drive the screw in a desired manner.

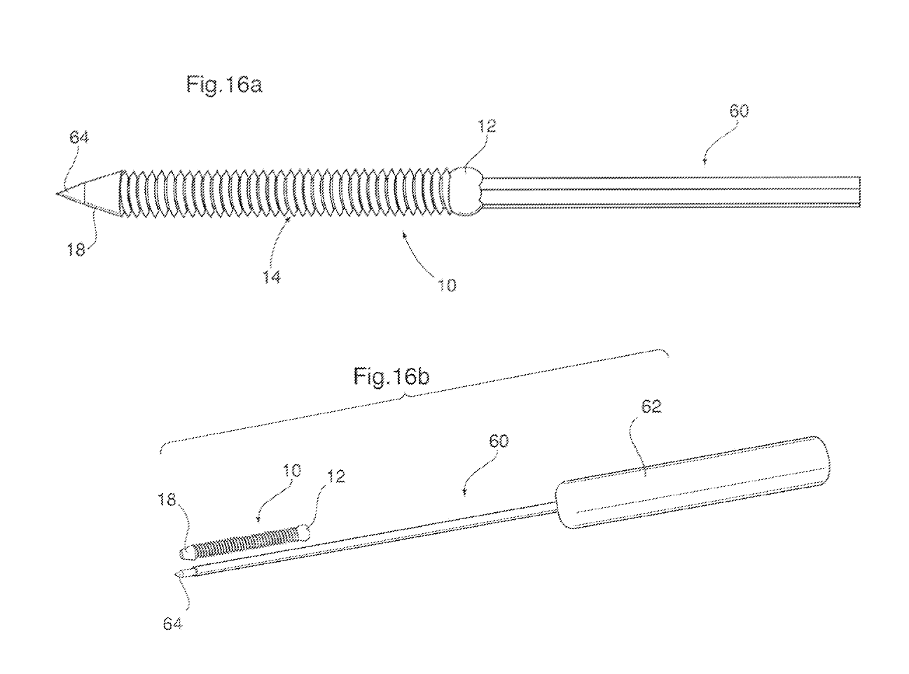

A further embodiment of the invention is illustrated in FIG. 16a, which shows, in combination, a screw 10, as described above, and an awl 60 that serves the function of the aforementioned driver. FIG. 16b illustrate the combination when separated. As shown in FIGS. 16a and 16b, the awl 60 includes a handle 62 and at least a hexagonal outer portion 62 at its distal portion or its proximal (i.e. handle) portion. In this way, the awl 60 can engage a cooperating opening in the head 12, the bone engaging element 18, as described above, or a combination of the two. As shown in FIGS. 16a and 16b, the awl 60 further includes a distal tip 64 that extends beyond the bone engaging element 18 when the screw 10 is combined with the awl prior to implanting the screw 10. The distal tip 64 may be provided with a point and/or a cutting edge, thereby allowing the awl to function as a piercing tool to facilitate positioning of the screw during implantation. Alternatively, the distal tip 64 may serve as a drill bit or drilling mechanism, to provide a borehole drilling function during implantation of the screw 10. Thus, as will be understood, the combination of the awl and the screw, as shown and described, allows the surgeon to combine the screw 10 with the awl 60 and, by rotating the awl, to implant the screw 10 in one step. Once implanted, the awl may be extracted.

The screw of the invention may be manufactured as a unitary body or multiple, separate sections that are then assembled or connected to form the screw. In one embodiment, the screws of the invention may be machined from a hollow rod, such as a titanium rod (or a rod from any material acceptable for implantation).

In another embodiment, as shown in FIGS. 11a and 11b, the screw of the invention 10 may be formed of three separate elements namely, a body 14, a bone engaging element 18, located at the distal end 13, and a head 12, located at the proximal end 11. FIG. 11a shows these components in the assembled state wherein they are joined to form the screw 10. FIG. 11b shows these components in an unassembled or exploded form. The components forming the screw may be joined by various means as known in the art. For example, the components may be joined by welding (such as, for example, using a solid state or “cold welding” process, or a fusion welding process), by a friction fit or by any other metal connecting methods. In one aspect, the body 14 may be provided with reinforced terminal ends 44 and 46, for attaching the head 12 and the bone engaging element, respectively. In such case, the head 12 and the bone engaging element 18 would be provided with stems shown at 48 and 50, respectively, which are preferably insertable into respective reinforced terminal ends 44 and 46 of the body 14. This arrangement would provide a desired contact surface area for securing the components together. In one aspect, the respective reinforced end of the body and the stems 48 and 50 may be provided with cooperating threading on opposing surfaces so as to allow each of the head and the bone engaging element 18 to be screwed on to the body 14. It will be understood that this manner of assembly may be used with a body that comprises a threaded cylinder as opposed to an open helix.

FIG. 13 illustrates the bone engaging element 18 as well as the preferred hexagonal lumen 51 for engaging the distal end of a driver. FIGS. 14a to 14c illustrate variations in the head 12. In FIG. 14a, for example, the head 12 is designed to receive the rod of a known spinal stabilizing structure. FIG. 14c illustrates a head 12 having a hexagonal shaped lumen adapted to receive a correspondingly hexagonal shaped driver. As discussed above, this form of the head 12 may be used for screws that are driven exclusively or partially from the proximal end of the screw.

FIGS. 20 to 22 illustrate a further embodiment of the bone engaging element, identified as 80, that is adapted to provide a bone cutting function as well. In this case, the bone engaging element may be referred to as a bone cutting edge or element. As described above, such bone cutting function may serve, in one aspect, to allow the screw to be “self tapping” or “self boring”. That is, rotation of the screw comprising such bone engaging element 80 would serve to drill the bone in contact therewith. This would allow the screw to be driven into the bone without the need for a borehole being provided. Alternatively, the bone engaging element 80 may equally be used with the provision of a borehole and wherein such element 80 serves to adapt the size of the borehole to accommodate the screw to which it is attached. In such cases, it will be understood that the borehole may serve as a “pilot hole” to assist in guiding the screw into the bone at or to a specific location. As shown in FIG. 20, the bone engaging element 80 is provided with a distal end 82 and a proximal end 84. As will be understood, the terms “distal” and “proximal” will have the same meanings as provided above. The distal end 82 functions as a cutting edge by means of a plurality of cutting elements 86 extending generally axially away in the proximal to distal direction. The cutting elements 86 may comprise any shape or orientation sufficient to function in cutting bone. Various modifications of the cutting edge will be apparent to persons skilled in the art. In one example, as illustrated in FIG. 20, the cutting edge may be formed by cutting notches, such as “V” shaped notches 88, into the distal end of the bone engaging element 80. To further assist in the cutting function of the element 80, longitudinally extending grooves 90 may be provided over the length of the element 80. As illustrated in FIG. 20, the bone engaging element 80 is shown as a separate element from the body of the screw. However, it will be understood that the same cutting edge as shown may equally be provided on a screw having a unitary structure. FIG. 20 illustrates the bone engaging element 80 having a stem 50, similar to that described above, which serves to attach such element 80 to a helical body portion when forming the screw of the invention.

FIGS. 21 and 22 illustrate an embodiment of the bone engaging element 80 having a lumen 51 for receiving a driver (not shown) as described above. In the embodiment illustrated, the lumen 51 is provided in a hexagonal shape, adapted to receive a complementary shaped driver and, thereby, function as a driver engaging means or device, as discussed previously. As described above, various other geometries would be possible for achieving the desired coupling between the screw and the driver. FIGS. 21 and 22 also illustrate the lumen 51 extending completely through the length of the element 80. Such a structure would, for example, be adapted to receive a driver completely there-through. In such example, the driver may comprise an awl as described above in reference to FIGS. 16a and 16b. As will be understood, the combination of an awl having a cutting tip, as described above, and a bone engaging element 80, having a cutting edge at its distal end 82, may allow the screw of the invention to be implanted into bone without the need for a borehole or pilot hole. That is, during implantation, the awl may be first coupled to a screw, having the bone engaging element 80, and can then be used to create an initial hole into the bone. The cutting edge of the bone engaging element 80 would then serve to increase the diameter of such hole to accommodate the body of the screw. As indicated above, the rotation of the awl will cause rotation of the screw as well due to the coupling between the driver and the screw.

FIGS. 20 to 22 illustrate the bone engaging element 80 having a lumen 51 adapted to function as a driver engaging means, that is, adapted to receive and be rotated by a driver. However, as discussed above, the driver engaging means can be provided at one or more other sections along the length of the screw.

As will be understood by persons skilled in the art upon reviewing the present description, the screw of the present invention offers a number of advantages. For example, it will be appreciated that the body 14 of the screw, due to its open helical structure, allows for an increased amount of screw surface area that contacts the adjacent bone. That is, as compared to known screws comprising a solid rod with a threaded outer surface, the screw of the invention allows a greater surface area of the “thread” to contact bone tissue. This therefore increases the total amount of the screw that contacts bone upon implantation. Further, the open helical structure of the invention also enables bone to grow through the body of the screw thereby increasing the degree of grip by which the screw is held within the bone. In another aspect, the interior of the screw may be filled with various compositions known in the art for promoting or enhancing bone in-growth and/or bone cementing compositions. For example, the interior may be filled with bone cementing or substitution substances, such as poly(methyl methacrylate) (PMMA), substances for inducing or enhancing bone growth, such as bone morphogenetic proteins (BMPs), or any combination(s) thereof. In such cases, it will be understood that the open nature of the screw of the invention facilitates the incorporation of such compositions.

In addition, the open helical structure also provides the screw with a degree of elasticity thereby allowing, for example, the head region of the screw to be laterally displaced or bent in relation to the body. As mentioned previously, studies of prior art pedicle screws have found that a high shear stress is developed at the junction of the head and the screw body post implantation. Thus, as discussed above, in cases where, after implantation, adjacent vertebral structures are displaced, the helical structure of the screw would be capable of withstanding the stresses applied thereto.

As discussed above in reference to FIG. 4, the screw of the present invention may comprise one or more helixes combined together to form the body. Various figures of the present application depict a single helix structure while FIG. 4 illustrates a double helix structure. As mentioned above, a multi-helix structure is also encompassed within the scope of the present invention. In a further aspect of the invention, a “hybrid” structure for the screw is contemplated though not shown in the figures. In such structure a portion of the length of the screw may comprise a solid cylinder that is typical of known bone screws, while the remaining portion comprises an open helical structure as taught herein. With this type of hybrid structure, the screw, where solid, would be provided with a stiffer portion as compared to the open helix portion. Thus, in one embodiment, the open helix portion may comprise only that portion of the screw that is implanted while the portion of the screw that is left outside of the bone or that comprises the proximal end comprises a solid screw. As will be understood, with such a structure, the portion that is implanted in bone will benefit from the advantages of an open helix structure as described above, while the portion of the screw that is external of bone, is provided with greater stiffness so as to improve its function, for example, in supporting the spinal stabilization system. Similarly, a portion of the proximal end of the screw may be formed as a solid but threaded section, while the body and distal portions are formed in the aforementioned open helical structure.

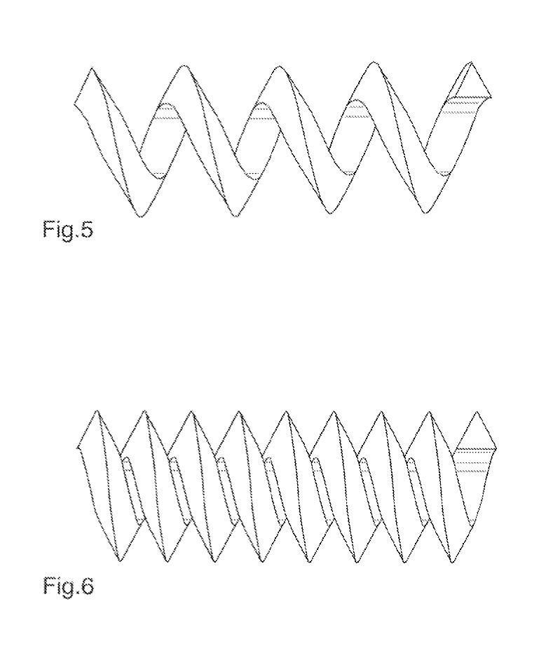

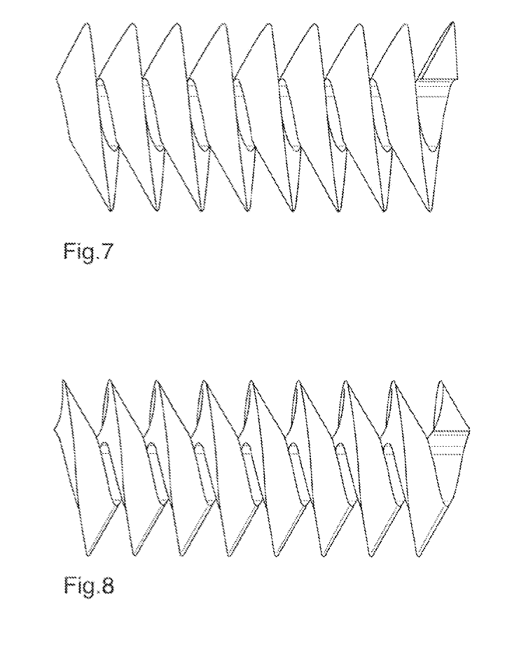

FIGS. 5 to 8 illustrate various different structures for the helix that forms the body of the screw. As will be noted, the screw of the invention may be provided with a threading of any type of profile configuration as will be apparent to persons skilled in the art. The term “profile configuration” is meant to describe various characteristics of screw threading as known in the art such as, inter alia, pitch, thread width, diameter (inner and outer), angular deflection of threading etc. It will also be appreciated that the invention is not limited to any one of the aspects described above and that any combination thereof may be used.

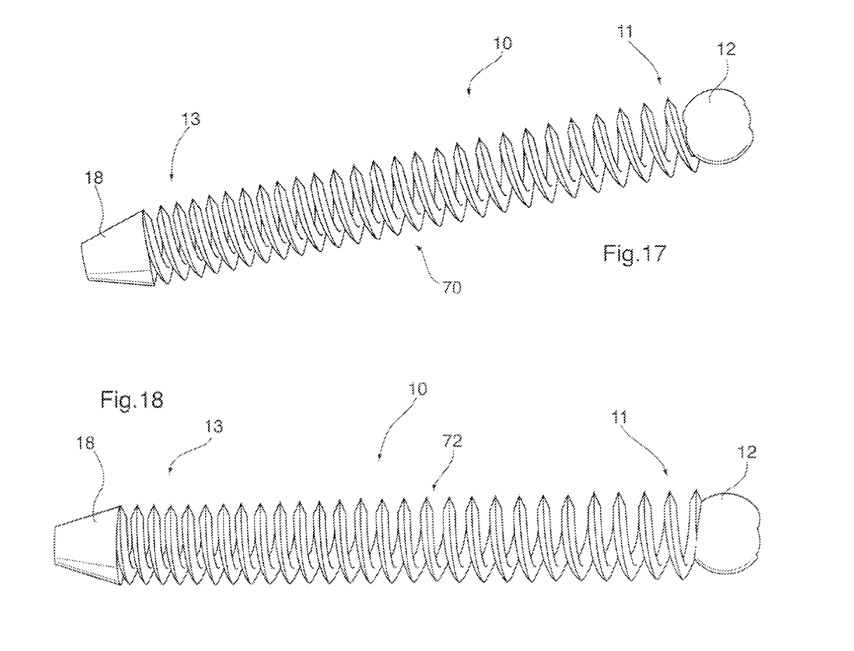

One example of the variability in the pitch of the thread forming the helical screw body is illustrated in FIG. 17. As shown, in one aspect of the invention, a screw 10 comprises a proximal end 11, including a head 12, and a distal end 13 including a bone engaging element 18, similar to those described above. However, in this aspect of the invention, the body 70 is provided with an open helical structure that varies in “pitch”, or the spacing of the threads comprising the helix as measured along the longitudinal axis of the screw. As will be understood, the pitch of the helix is deemed to be lower at a given point than another when the number of threads per unit length is higher at such point (i.e. the spacing between adjacent threads is lower). In the screw shown in FIG. 17, it is noted that the pitch of the helix is lower towards the distal end 13 of the screw as compared to the proximal end 11. As will be understood by persons skilled in the art, a helix having a lower pitch would provide the helix with greater stiffness. Thus, in the example illustrated in FIG. 17, the distal portion of the helix, by being provided with a lower pitch, would be stiffer than the proximal portion, which has a higher pitch. In addition, it will be understood that a single rotation of the screw shown in FIG. 17 will result in a difference in screw surface to bone contact as between the distal and proximal ends. For example, in the case of the screw shown in FIG. 17, with one rotation thereof, the portion of the helix at the distal end 13 will rotate to a greater degree than the portion at the proximal end 11 as a result of the difference in pitch. As will be understood, a screw according to the invention can be provided with the aforementioned pitch reversed, thereby resulting in the proximal portion of the screw being stiffer than the distal portion. It will also be understood that a number of variations in the pitch of the helix may be provided in order to provide the resulting screw with any desired variation in stiffness along its length or at certain discrete sections. The present invention is not limited to any one pitch or pitch design.

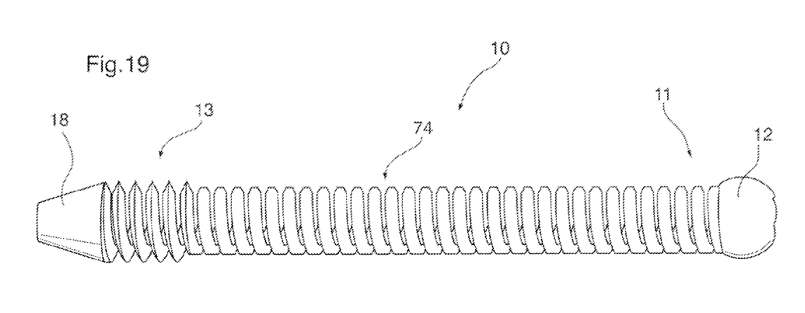

A further aspect of a screw according to the invention is illustrated in FIG. 18 wherein a screw 10 is provided with a body 72 having a variable pitch helix as described above. That is, the pitch of the helix at a region of the distal end 13 is less than the pitch at a region of the proximal end 11. However, in this embodiment, the screw is also provided with a taper wherein the diameter of the screw at the distal end 13 is less than the diameter at the proximal end 11. Such variability in diameter along the longitudinal axis also serves to vary the stiffness characteristics of the screw. It will be understood that any degree of taper, or lack thereof, may be used with the screws of the invention. FIG. 19 illustrates a variation of the screw 10 wherein the portion of the screw body 74 at the distal end 13 is provided with a greater diameter than the portion at the proximal end 11.

The screws and screw components of the present invention can be made of any material as will be known to persons skilled in the art. For example, the elements of the invention may be made of: metals or metal alloys such as stainless steel, titanium, titanium alloys, nickel-titanium alloys (such as Nitinol™), cobalt-chrome alloys; plastic and/or thermoplastic polymers (such as PEEKT™); carbon fiber; or any other material, or combination of materials, commonly associated with bone screws. It will also be understood that the surface of the screws and screw components of the invention may optionally be coated with any known substances for improving their placement or adhesion within the bone. For example, in one embodiment, the outer surface of the screw, or at least that portion that will be in contact with bone after implantation, may be coated with hydroyapatite to promote osseointegration of the screw and, thereby, inhibit or prevent screw pullout.

The open helical structure of the invention allows for the screw to be compressed or expanded prior to insertion into the bone. For example, as discussed above in reference to FIG. 9, in one embodiment, the driver 40 is inserted axially into the lumen of the open helix screw 10, extending through the head 12, to engage the bone engaging element 18. In such embodiment, the proximal portion of the driver can engage the head 12 as well. In such orientation, rotation of the driver drives rotation of the screw at both the distal and proximal ends. However, in addition to such dual rotation, it is also possible to apply a distracting force through the driver 40 so as to slightly lengthen or stretch the helix of the screw along its longitudinal axis. In such state, when the distracted screw is placed into, for example, a fractured bone the release of the distracting force through the driver, and the resilient characteristic of the helix, will force the screw to return to its original state. This tendency will cause the screw to shorten in the bone thereby resulting in compression of the fractured fragments against each other. Such compressive state is known to enhance bone healing. It will be understood that, in a similar manner, the screw of the invention can be compressed prior to implantation, thereby serving to provide a distractive force on the bone when implanted.

In a further aspect, the driver 40 may be used to “unwind” or “wind-up” the helix of the screw to provide the aforementioned compressive of distractive forces. In this aspect, one end of the screw would be held stationary, preferably when loaded on the driver, while the opposite end is rotated. As will be understood, such rotation of one end results in a twisting or torquing of the screw. In the result, the screw will be pre-loaded with either a compressive or distractive force prior to implantation. When the driver is removed, after implantation of the screw into the bone, the helix will tend to resume its normal shape thereby imparting the desired forces between the distal and proximal ends of the screw. Various methods may be used to twist the screw. For example, in one aspect, the driver may be provided with a means to rotate the head of the screw in either direction while preventing rotation of the distal end. As discussed above, one aspect of the invention provides for the distal ends of the driver and the screw to be complementary in shape (e.g. hexagonal) and, in such arrangement, it will be understood that this would be one way of preventing rotation of the distal end of the screw.

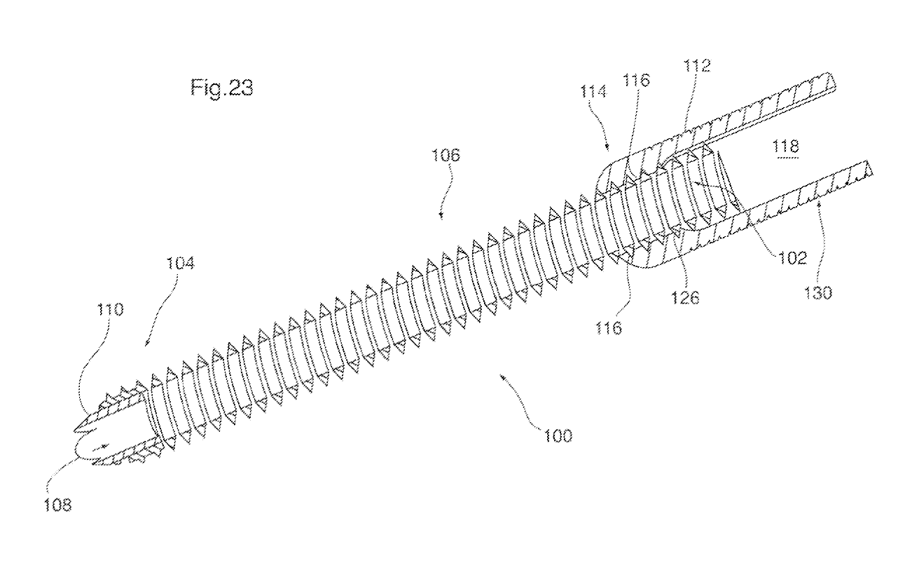

A further aspect of the invention is illustrated in FIGS. 23 to 28 wherein a unique combination of separate screw and head is illustrated. In this aspect, the screw 100 is generally the same as that described previously. In particular the screw 100 includes a proximal end 102, a distal end 104 and a body portion 106 extending there-between. In one embodiment, the body portion 106 comprises a hollow structure having a central bore 108. In the embodiment shown in FIG. 23, the body portion 106 comprises an open helical structure, as described above, composed of one or more helical elements arranged to form the threading of the screw. Again, by “open helical structure” or “open helix” it is meant that the spaces between each thread are open to a central bore 108 of the screw, similar to a “corkscrew”. As described previously, the distal end 104 is adapted to engage bony material during the implantation step. For this purpose, the distal end 104 may be provided with a bone engaging element 110, such as described above. Alternatively, particularly in the case where the body portion 106 is an open helix, the distal end 104 may comprise a sharpened ends of the one or more helical elements.

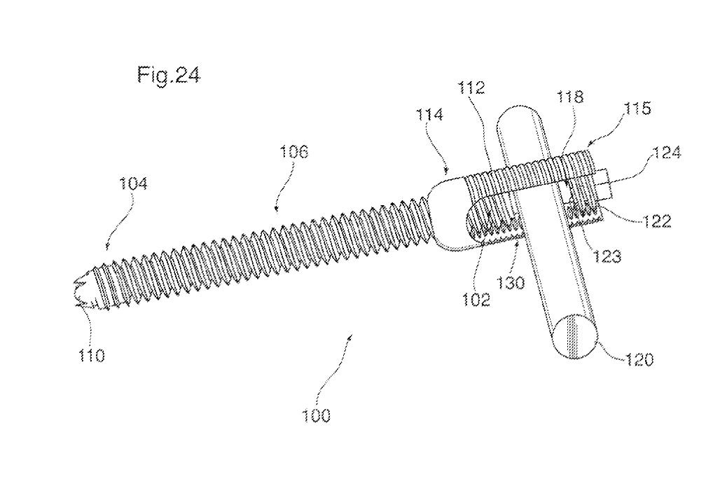

In the embodiment of the invention as illustrated in FIGS. 23 and 24, the head 112 of the screw 100 comprises a generally cylindrical hollow body having a first, distal end 114 that cooperates with and engages proximal end 102 of the screw 100. For example, in the embodiment shown, the internal bore of the distal end 114 of the head 112 is provided with threads 116 that cooperate with the threads formed or provided at the proximal end 102 of the screw. In this manner, the head 112 can be threaded onto the proximal end 102 of the screw 100 and positioned at any location along the length thereof.

As shown in FIG. 24, the head 112 of the illustrated embodiment may be preferably provided with a slot 118 extending there-through. The slot 118 is adapted to receive a rod 120 or other such apparatus typically used for spinal stabilization as known in the art. The internal bore of the second, or proximal end 115 of the head 112 would also preferably be provided with threads that are adapted to receive a locking nut 122. The locking nut 122 would typically have a bearing end 123 and a driving end 124. The bearing end 123 is adapted to bear against the outer surface of the rod 120 and thereby secure the head 112 to the rod 120 once the desired relative positioning has been established. The driving end 124 of the locking nut 122 may be adapted in any manner to receive a driving tool. For example, as shown in FIG. 24, the driving end 124 may be provided with a hexagonal shape to receive a suitably shaped tool. It will be understood that the configuration of the driving end 124 is variable.

One advantage of the embodiment shown in FIGS. 23 to 28 lies in the adjustable positioning of the head 112 with respect to the screw 100. With known bone screws, such as pedicle screws and the like, the heads provided on such screws are generally fixed to the end of the screw shaft. Such design does not allow for adjustment of the head position. However, with the embodiment of FIGS. 23 to 28, the head 112 may be rotated or threaded to any position along the length of the screw 100. Once a desired position is reached, the head may be fixed to the screw 100 using a variety of methods. For example, the head may be secured or fixed to the screw 100 using a cold welding method or the head may be retained in position by a friction fit. Alternatively, any other means such as adhering etc. can be utilized for this purpose. Further, since the head 112 is threaded onto the screw 100, the amount of contact surface area between the head and the screw is large.

The locking nut 122 also serves to “lock” the screw 100, head 112 and rod 120 together. More specifically, as will be understood, a screw-rod spinal stabilization construct is formed when a screw 100, which comprises the bone anchoring device, secured to one vertebra is connected to another screw secured to an adjacent vertebra by means of a link. In one aspect, the link comprises the rod 120. To provide for a stable construct the screw-rod connection should preferably be rigid and not allow for any movement once the construct is “locked”. The head 112 serves to secure the screw 100 to the rod 120. As discussed above, this may be accomplished by a cold weld or a friction fit between the head 112 and screw 100 interface. A locking nut 122 may then be screwed onto the head 112 to secure the rod 120 to the head 112 and thereby to the screw 100. Such a “friction fit” may be accomplished by tightening of the locking nut 122. Such tightening increases the friction between the contact surfaces of the screw 100 and head 112. Further, since the rod 120 prevents further rotation of the head on the screw, the positioning of the head would be fixed. In addition, where the screw 100 comprises an open helix (i.e. a shaft-less screw) it is possible, according to the invention, to compress the portion of the screw thread contained within the slot 118 of the head 112. By compressing this portion of the screw thread, it will be understood that the head 112 is tightened against the screw 100. Furthermore, the force applied by tightening the locking nut 122 also serves to pull the head 112 against the rod 120. This therefore serves to essentially “lock down” the construct providing rigid fixation.

In another aspect, the sizing of the thread 116 provided on the head 112 can be tailored. For example, where the thread 116 closely or exactly corresponds to the threading provided on the screw 100, it will be understood that very little relative movement between the head 112 and the screw 100 is possible. Such an orientation results in a fixed angle screw. However, in some cases, it may be desired for the angle of the head to be adjusted along various axes. In such case, the thread 116 of the head 112 may be sized to allow a degree of relative movement between the head 112 and the screw 100. Such an orientation would be advantageous when considered against some known devices such as that taught in U.S. Pat. No. 7,314,467 wherein a system comprising a plurality of head designs are required depending on the angle required to receive a spinal stabilization rod.

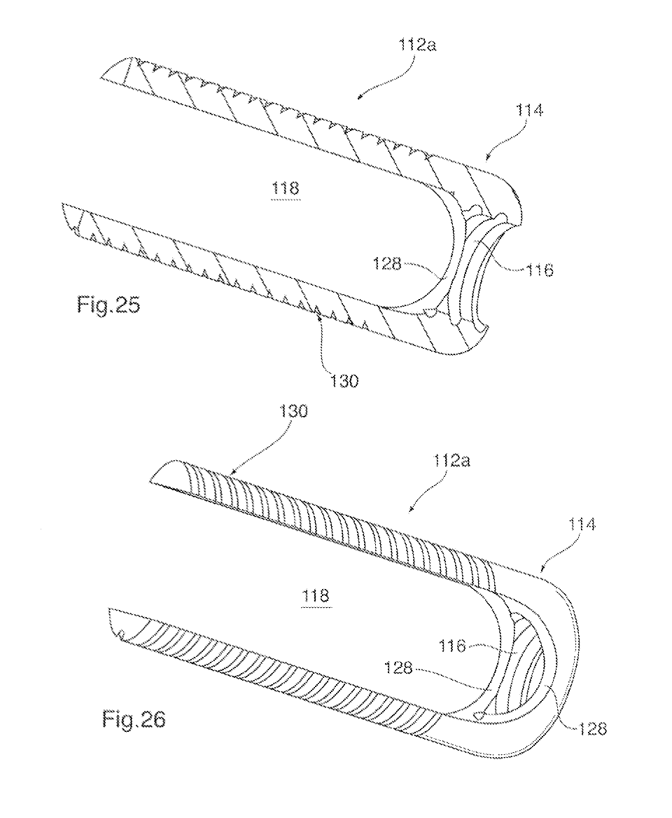

FIGS. 25 to 28 illustrate another embodiment of the head, identified as element 112a, which comprises a shorter distal end 114. That is, the amount of threading 116 provided on the head 112a to engage the screw 100 is less than that of the embodiment shown in FIGS. 23 and 24. In the result, the head 112a would be able to rotate more easily with respect to the screw 100 in a multiaxial manner. To further assist such movement, the threading 116 of the head 112a may also be rounded somewhat to allow a degree of relative mobility between the head 112a and the screw 100. In addition, the distal end of the slot 118 may also be provided with a curved surface such as that shown as element 128 in FIGS. 25 and 26. These features, either individually or in combination, allow the head 112a to “wobble” with respect to the screw 100 until such time as it is locked in position as described previously. This therefore allows the head to be positioned as needed to receive the rod prior to being locked.

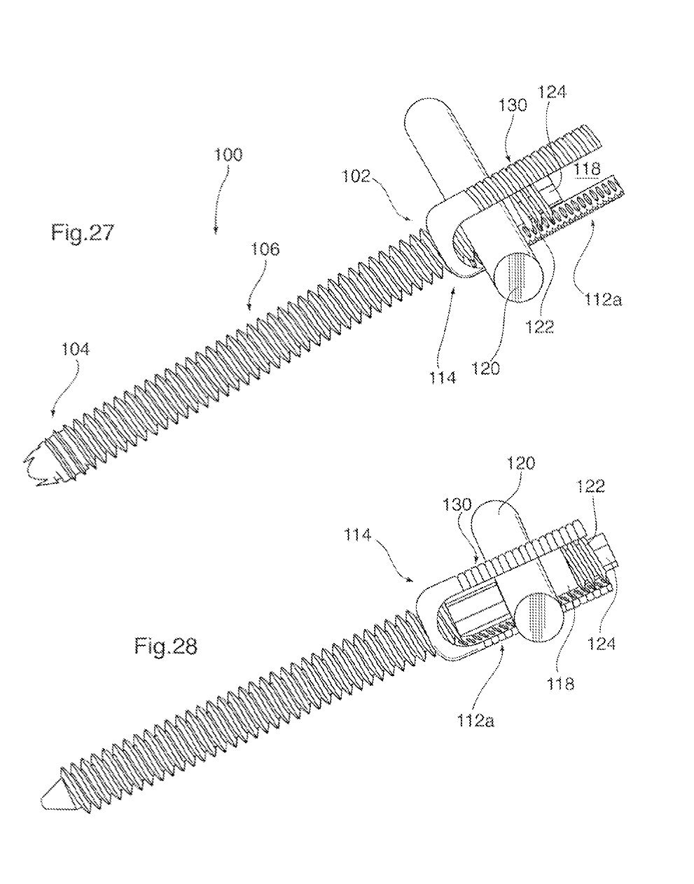

In the above description with respect to FIGS. 23 to 28, it will be understood that the body 106 and distal end 104 of the screw 100 may assume any of the aforementioned orientations. In a similar manner, although the above description has referred to the body of the screw being an open helix, it will be appreciated that the unique head 112 of the invention may be used with a solid screw as well. This feature is illustrated in FIGS. 27 and 28. As will be appreciated, the advantages offered by the head 112 or 112a, as described above, would apply equally to a screw having the aforementioned open helical shape (FIG. 27), a solid screw (FIG. 28) or a cannulated screw (not shown). As known in the art, a cannulated comprises screw shaft having a longitudinal bore.

As can be seen in comparing FIGS. 27 and 28, the manner in which the rod 120 is locked to the screw and head combination is generally the same.

As discussed above, a further advantage offered by the embodiment of FIGS. 23 to 28 is that the height of the head 112 could also be adjusted. This provides flexibility in instances where the anatomy might require it. This technique of fixation could be used for not only the open helix screws (or shaft-less screws) but also solid shaft or cannulated screws. As will be understood, in the latter case, the screw thread would not be compressible; however, the rod will still be compressed between the sold shaft of the screw and the locking nut 122. This technique would be useful for reducing spondylolisthesis.

In FIGS. 23 to 28, the head 112, 112a is shown as being “open” at the proximal end (which receives the locking nut 122). That is, the slot 118 is illustrated as extending completely through the proximal end. However, it will be understood that the invention is not restricted to such structure. It will be appreciated, for example, that the proximal end may be “closed” thereby providing the slot 118 with a desired finite length. The “open” proximal end would be understood to have the advantage of being able to receive a rod 120 axially into the slot 118. In the case of a “closed” proximal end, it will be understood that the rod 120 would need to be inserted or fed through the slot opening.

In another embodiment of the invention shown in FIGS. 23 to 28, the outer surface of the head 112, 112a may be provided with a threaded region 130 over which a screw cap (not shown) or other such element may be secured. In one aspect, the threaded region 130 may be provided only at the proximal end of the head so that the cap may be screwed over the outer surface of the head 112, 112a. As will be appreciated, including such a cap will serve to close and/or reinforce the proximal opening of the head and may also serve to prevent dislodging of the locking nut 122. It will be understood that the screw cap, or closure, can assume any shape to serve this purpose.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

1. A bone screw comprising:
an elongate body having a first end, a second end and a body portion extending there-between;
the body portion having an open helical structure, comprising at least one open helix, forming threads on the outer surface of the body portion, wherein spaces between the threads open into an axial bore extending through the body portion;
the first end including a head; and,
the second end including an anchoring portion adapted to engage bony material.

2. The bone screw according to claim 1 further comprising a first driver engaging element provided at the second end, said first driver engaging element being adapted to engage a driver for turning the bone screw.

3. The bone screw according to claim 2 wherein the head includes an opening extending into the axial bore of the body portion.

4. The bone screw according to claim 3 further comprising a second driver engaging element provided within the head, said second driver engaging element being adapted to engage the driver for turning the bone screw.

5. The bone screw according to claim 1, wherein said second end includes a bone cutting edge or element, for boring into bone during implantation.

6. The bone screw according to claim 5 wherein said second end includes a self-tapping element.

7. The bone screw according to claim 1, wherein the body portion, the first end and the second end form a unitary structure.

8. The bone screw according to claim 1, wherein said screw is formed of one or more sections comprising the body portion, the first end and the second end, and wherein such sections are adapted to be connected or joined together.

9. The bone screw according to claim 1, wherein a segment of the body portion adjacent at least one of the first or second ends comprises a solid, externally threaded cylinder, wherein spaces between the threads are closed.

10. The bone screw according to claim 1, wherein the head includes an axial bore with an internal thread and wherein said internal thread cooperates with the threads of the body portion, whereby the head is adapted to be secured to the body portion.

11. The bone screw according to claim 1, wherein the position of the head is adjustable axially along the length of the body portion.

12. The bone screw according to claim 11 wherein the head includes a threaded opening cooperating with the threading of the body portion.

13. The bone screw according to claim 12 further comprising a locking nut to lock the head in position with respect to the body portion.

14. The bone screw according to claim 13 wherein the head includes a cylindrical, threaded external surface adapted to receive a screw cap.

15. The bone screw according to claim 11 wherein the head is moveable along one or more axes with respect to the body portion.

16. The bone screw according to claim 1, wherein said screw comprises a pedicle screw.

17. The bone screw according to claim 16 wherein said head is adapted to connect to a spinal stabilization prosthesis.

18. A bone screw comprising:
an elongate body having a first, proximal, end, a second, distal, end and a body portion extending there-between;
the body portion comprising an externally threaded cylindrical rod with an axial bore extending longitudinally along at least a portion thereof;
the first end including a head with an opening extending into the bore;
the second end including an anchoring portion adapted to engage bony material; and,
a first driver engaging element provided at the second end, said first driver engaging element being adapted to engage a driver for turning the bone screw.

19. The bone screw according to claim 18 further comprising a second driver engaging element provided within the head, said second driver engaging element being adapted to engage the driver for turning the bone screw.

20. The bone screw according to claim 18, wherein said second end includes a bone cutting edge or element, for boring into bone during implantation.

21. The bone screw according to claim 20 wherein said second end includes a self-tapping element.

22. The bone screw according to claim 18, wherein the body portion, the first end and the second end form a unitary structure.

23. The bone screw according to claim 18, wherein said screw is formed of one or more sections comprising the body portion, the first end and the second end, and wherein such sections are adapted to be connected or joined together.

24. The bone screw according to claim 18, wherein the head includes an axial bore with an internal thread and wherein said internal thread cooperates with the threads of the body portion, whereby the head is adapted to be secured to the body portion.

25. The bone screw according to claim 18, wherein said screw comprises a pedicle screw.

26. The bone screw according to claim 25 wherein said head is adapted to connect to a spinal stabilization prosthesis.

27. The bone screw according to claim 18, wherein the position of the head is adjustable axially along the length of the body portion.

28. The bone screw according to claim 27 wherein the head includes a threaded opening cooperating with the threading of the body portion.

29. The bone screw according to claim 28 further comprising a locking nut to lock the head in position with respect to the body portion.

30. The bone screw according to claim 29 wherein the head includes a cylindrical, threaded external surface adapted to receive a screw cap.

31. The bone screw according to claim 27 wherein the head is moveable along one or more axes with respect to the body portion.

32. The bone screw according to claim 18, wherein said screw comprises a pedicle screw.

33. The bone screw according to claim 32 wherein said head is adapted to connect to a spinal stabilization prosthesis.

34. A spinal stabilization system comprising one or more bone screws according to claim 1 and spinal stabilization prostheses adapted to be connected to said screws.

35. The system according to claim 34 wherein said one or more bone screws are pedicle screws and wherein said prostheses are spinal stabilization rods.

36. A method of implanting a bone screw comprising:
a) providing a bone screw having:
an elongate body having a first, proximal, end, a second, distal, end and a body portion extending there-between;
the body portion comprising: (i) an externally threaded cylindrical rod with an axial bore extending longitudinally along a substantial portion of said body; or (ii) an open helix structure, wherein spaces between the threads open into an axial bore extending through the body portion;
the first end including a head with an opening extending into the hollow cavity;
the second end including an anchoring portion adapted to engage bony material; and,
the second end including a first driver engaging element;
b) providing a driver having a first end adapted to engage the first driver engaging element;
c) placing the second end of the screw against a bone structure;
d) rotating the driver thereby rotating the second end of the screw; and,
e) driving the screw into the bone structure.

37. The method according to claim 36 wherein the head includes a second driver engaging element for receiving said driver and wherein step (d) comprises rotating the first and second ends of the screw.
